(19) United States
(12) Patent Application Publication
(10) Pub. No.: US 2021/0268224 A1
(43) Pub. Date: Sep. 2, 2021

(54) A HUMIDIFICATION ASSEMBLY

(71) Applicant: Fisher & Paykel Healthcare Limited, East Tamaki, Auckland (NZ)

(72) Inventors: Craig Karl White, Auckland (NZ); Gareth Thomas McDermott, Auckland (NZ); Cameron Leslie Matthews, Auckland (NZ); Shane Terry Massey, Auckland (NZ)

(21) Appl. No.: 17/258,327

(22) PCT Filed: Aug. 30, 2019
(86) PCT No.: PCT/IB2019/057304

§ 371 Date: Jan. 6, 2021

Related U.S. Application Data
(60) Provisional application No. 62/724,995, filed on Aug. 30, 2018.

(51) Int. Cl. A61M 16/16; A61M 16/06; A61M 16/08

(52) U.S. Cl. CPC A61M 16/162; A61M 2205/3334; A61M 16/0816; A61M 16/0672

(57) ABSTRACT

This invention relates to a humidification assembly or kit for use in a respiratory support system for delivering humidified gas to a user or patient. The assembly or kit comprises a fluid reservoir, and a humidification chamber. The fluid reservoir and the humidification chamber are provided in fluid connection with each other, wherein a connection between each of the fluid reservoir and the humidification chamber provides a pre-determined force of retention and/or resistance to disconnection or is a permanent type connection.

### TECHNICAL FIELD

This disclosure relates to various humidification assemblies, fluid containers, conduits, and humidification chambers, and particularly to permanent connections between fluid reservoirs and humidification chambers. The humidification assemblies include connections that are permanent or deliberately difficult to remove, where removal may cause damage to the connection or assembly components. The disclosure also relates to connectors to facilitate the permanent or enduring connection of fluid reservoirs, such as water bags, to humidification chambers.

### BACKGROUND ART

Known fluid reservoirs and accessories such as humidifier chambers or infusion pumps have a conduit or tubing that is free to connect to other fluid reservoirs or accessories. This is because permanent connections at both ends of a conduit is usually not desirable. A benefit of known fluid reservoirs not having permanent connections is that the conduit (and patient) often needs to be plugged into a series of successive bags of the same or different fluids (such as water, medicament, or intravenous (IV) fluids). Thus, in this case, removable connections are provided at either end of the conduit and are preferred for optionality and choice in which fluid to provide to a patient at a given time, without the need to clean the administration equipment from the prior fluid or to provide a new set of administration equipment.

However, for humidifiers in an operating theatre situation, this is not necessarily the case. In this situation, water is usually the only fluid in communication with the humidification chamber.

Using known fluid bags, there exists a risk that a user, such as a nurse, clinician, or other medical professional, in a theatre situation, could accidently spike an incorrect fluid bag for an accessory, for example saline for the humidifier or water for an infusion pump or IV drip. If this accidental spiking were to occur in theatre, where it may be likely to as it is a busy and/or stressful environment, then an IV fluid such as saline could be introduced into the humidification chamber of a humidifier, and conversely distilled water could be introduced into a patient intravenously.

This issue is compounded because hypotonic water bags and saline bags look very similar. Another compounding factor is that there are no standards in terms of labelling of different fluid bags. In some countries, the wording for saline bags is blue in colour, but a blue wording could be used for hypotonic water in other countries.

Saline is a commonly used intravenous fluid. Typically, normal saline is a sodium chloride solution at 0.9% concentration. This concentration is close to the concentration in blood so saline is isotonic. If saline is accidentally used with a humidification system then when water evaporates, salt is left behind. This salt can cause problems with the function of the humidification system as it may get into and damage component parts, degrade sensors etc. For example, salt build up has shown to impede sensor readings and valve functions, which can result in overfilling of a humidification chamber. This could become a safety hazard in the operating theatre/room.

If hypotonic water is introduced into the patient instead of the intended saline, then the patient's blood can become hypotonic, with too low a solute concentration, and hemolysis of the red blood cells and/or cytolysis of other cells may occur. Hemolysis is the rupturing of red blood cells, and it can result in decreased oxygen supply to the rest of the body.

As outlined above, commonly used hypotonic water and saline bags look similar, thus increasing the risk of mixing these bags, and administering an incorrect bag to a patient (i.e. hypotonic water in IV, and/or saline in humidification). Hypotonic water already exists in bottles in theatre for other reasons, and these bottles are easily discernible from IV bags.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

### SUMMARY

It is an object of this disclosure to provide a fluid reservoir permanently connected to a conduit (tubing), in turn permanently connected to a humidification chamber, or a fluid reservoir in a permanent connection with a humidification chamber (such a permanent connection being provided in a manner such that the connections provide for a sufficient resistance to disconnection or a connection which is severed upon disconnection) which goes at least some way towards overcoming one or more of the above mentioned problems or difficulties, or to provide the industry/public with a useful choice.

Hypotonic water already exists in bottles in theatre for other reasons, and these bottles are easily discernible from IV bags. This invention allows this hypotonic water to be used to fill/refill a water bag connected to a humidification chamber. This invention removes the need to have the fluid bags containing hypotonic water in theatre, because pre-existing bottles of water may be used to fill up the water bag of this invention instead. Removing these hypotonic water bags would prevent any confusion and thus remove the risk of putting water into the patient instead of saline, and saline into the humidifier instead of water.

Therefore, this invention provides an ideal humidification system for a busy and/or stressful theatre environment that prevents saline accidentally being delivered to the humidifier and water accidently being delivered to the patient.

In accordance with at least one of the embodiments disclosed herein, a humidification assembly or kit for use in a respiratory support system for delivering humidified gas to a user or patient comprises, a humidification chamber, a fluid reservoir for supplying fluid to the humidification chamber, and wherein the humidification chamber and fluid reservoir are permanently connectable via a tubing.

In a first aspect there is provided a humidification assembly or kit for use in a respiratory support system for delivering humidified gas to a user or patient, comprising:
a fluid reservoir, and
a humidification chamber,
wherein the fluid reservoir and the humidification chamber are provided in fluid connection with each other, and
wherein, a connection between each of the fluid reservoir and the humidification chamber provides a pre-determined force of retention and/or resistance to disconnection.

The connection between each of the fluid reservoir and the humidification chamber may provide for a pre-determined force of retention and/or resistance to disconnection such that said connection is destructively severed upon application of a disconnection force above said pre-determined force.

A part or component of the humidification assembly or kit may be destroyed when the connection is subject to a disconnection force above said pre-determined force.

In a second aspect there is provided a humidification assembly or kit for use in a respiratory support system for delivering humidified gas to a user or patient, comprising:
a fluid reservoir,
a humidification chamber,
wherein in the fluid reservoir and the humidification chamber are provided in permanent fluid connection with each other.

In a third aspect there is provided a humidification assembly or kit for use in a respiratory support system for delivering humidified gas to a user or patient, comprising:
a humidification chamber configured for receiving a source of humidification fluid,
a fluid reservoir configured for providing the source of humidification fluid for supply to the humidification chamber, and
wherein a permanent connection is provided for a fluid connection between the fluid reservoir and the humidification chamber.

A device may provide for a fluid connection from the fluid reservoir and the humidification chamber.

The device providing for the fluid connection may be a conduit.

In a fourth aspect there is provided a humidification assembly or kit for use in a respiratory support system for delivering humidified gas to a user or patient, comprising:
a humidification chamber configured for receiving a source of humidification fluid,
a fluid reservoir configured for providing the source of humidification fluid for supply to the humidification chamber,
a conduit provided as an interconnect between the humidification chamber and the fluid reservoir configured for providing the source of humidification fluid, and
wherein, a connection is provided as a permanent connection between each of:
   the conduit and the humidification chamber, and
   the conduit and the fluid reservoir.

In a fifth aspect there is provided a humidification assembly or kit for use in a respiratory support system for delivering humidified gas to a user or patient, comprising:
a humidification chamber configured for receiving a source of humidification fluid,
a fluid reservoir configured for providing the source of humidification fluid for supply to the humidification chamber,
a conduit provided as an interconnect between the humidification chamber and the fluid reservoir configured for providing the source of humidification fluid, and
wherein, a connection is provided with a pre-determined force of retention and/or resistance to disconnection between each of:
   the conduit and the humidification chamber, and
   the conduit and the fluid reservoir.

The following features may be provided in combination with any one or more of the above aspects or features as disclosed herein.

The connection may be one or more of:
configured such that, the or a conduit providing for the fluid communication is resistant to, or unable to be disconnected,
configured such that disconnection results in damage to the fluid communication,
configured such that separation or disconnection of the connection serves to destructively sever the connection,
configured such that separation or disconnection of the connection is destructive of one or more components or parts which formed the connection,
configured such that separation or disconnection of the connection serves to prevent a subsequent re-establishment of a connection.

A first end of the conduit may be connected or coupled to the fluid reservoir, or a component connected to or coupled to the fluid reservoir.

A second end of the conduit may be connected or coupled to the humidification chamber or a component connected to or coupled to the humidification chamber.

The conduit may be provided as the fluid connection between the fluid reservoir and the humidification chamber, and may be configured for providing the source of humidification fluid.

The conduit may be provided as a fluid interconnect between the humidification chamber and the fluid reservoir configured for providing the source of humidification fluid, and the connection may be established between each of:
the conduit and the humidification chamber, and
the conduit and the fluid reservoir.

The connection between the conduit and humidification chamber may be made by one or more of:
a barbed-type connector,
a structural formation providing for a physical inter-connect,
an interference or friction fit or push-fit,
an adhesive,
solvent bonding,
heat sealing,
a threaded connection.

The connection between the conduit and said fluid reservoir may be made by one or more of:
a barbed-type connector,
a structural formation providing for a physical inter-connect,
an interference or friction fit or push-fit,
an adhesive,
solvent bonding,
heat sealing,
a threaded connection.

A fluid reservoir end of said conduit may comprise a fluid reservoir connector to provide the connection between said device or said the conduit and the fluid reservoir.

A humidification chamber end of said conduit may comprise a humidification chamber connector to provide the connection between the conduit and the humidification chamber.

The conduit may comprise:
a fluid reservoir connector at a fluid reservoir end of said conduit, and
a humidification chamber connector at a humidification chamber end of said conduit,
wherein each of said fluid reservoir connector and said humidification chamber connector may provide separately made connections between the conduit and the fluid reservoir and the conduit and the humidification chamber.

The fluid reservoir connector may be connected to the fluid reservoir end of said conduit.

The fluid reservoir connector may provide a hermetic connection between the conduit and the fluid reservoir.

The fluid reservoir connector may be connected to the fluid reservoir end of said conduit by one or more of:
a barbed-type fit,
a structural formation providing for a physical inter-connect,
an interference or friction fit or push-fit,
an adhesive,
solvent bonding,
heat sealing,
a threaded connection.

The humidification chamber connector may be connected to the humidification chamber end of said conduit.

The humidification chamber connector may provide a hermetic connection between the conduit and the humidification chamber.

The humidification chamber connector may be connected to the humidification chamber end of said conduit by one or more of:
a barbed-type fit,
a structural formation providing for a physical inter-connect,
an interference or friction fit or push-fit,
an adhesive,
solvent bonding,
heat sealing,
a threaded connection.

The fluid reservoir connector and/or the humidification chamber connector may comprise a lead-in portion substantially at or toward one end of said connector, said lead-in portion to facilitate a connection between:
the fluid reservoir connector and the fluid reservoir, and/or
the humidification chamber connector and the humidification chamber.

The lead-in portion may be a tapered region.

The lead-in portion may be shaped so as to facilitate the subsequent establishment of connection.

The tapered region may be tapered to provide for a relatively narrowed neck of a relatively smaller diameter to receive said conduit or to be received by the fluid reservoir or the humidification chamber.

The fluid reservoir connector and/or the humidification chamber connector may be substantially flexible or substantially conformable or substantially non-rigid.

The fluid reservoir connector and/or the humidification chamber connector may be substantially inflexible or substantially non-conformable or substantially rigid.

The fluid reservoir connector and/or the humidification chamber connector may be substantially rigid, and optionally may be formed of a polypropylene.

The fluid reservoir connector may be a substantially longitudinal component comprising a first end configured to be received by the fluid reservoir, and a second end configured to be received by the conduit.

The fluid reservoir connector may comprise a lead-in portion at or toward the first end.

The fluid reservoir connector may comprise a barb-type portion at or toward the second end.

The barb-type portion may be configured to retain said conduit once said connection is established.

At least one radially outward projection may extend from an outer surface of the fluid reservoir connector, said at least one radially outward projection may be located intermediate of the lead-in portion and the second end.

The at least one radially outward projection may be or comprise of a barb-type arrangement, configured to retain said fluid reservoir once said connection is established.

A leading edge or leading surface of said at least one outward projection may be leading with respect to an edge or surface of the projection(s) which is/are to preferentially make contact with said fluid reservoir.

A collar may be located intermediate of the at least one radially outward projection and the second end.

The collar may provide for at least two seating surfaces, a first of said seating surfaces provided to substantially face toward the first end and a second of said seating surfaces provided to substantially face toward the second end.

Each seating surface of the at least two seating surfaces may be provided as a continuous or discontinuous surface.

The collar may be an annular collar.

The collar may extend radially outwardly from the outer surface of the fluid reservoir connector, the collar dimensioned to have a longitudinal thickness configured to separate a terminal end of the conduit from a fluid reservoir connection point.

The collar may be a thickened band region of the fluid reservoir connector.

A leading edge or leading surface of said at least one outward projection may be leading with respect to an edge or surface of the projection(s) which is/are to preferentially make contact with said fluid reservoir.

The at least one radially outward projection may extend outwardly of the outer surface to a substantially lesser extent or diameter than said collar.

The collar may extend substantially radially outwardly of the outer surface to a substantially greater extend, extent, or diameter than said outward projection.

The barb-type portion may be located substantially at or toward said second end, and may be located intermediate of the collar and the second end.

The external diameter of the connector between the second end of said connector and the collar may comprise a first longitudinal length diameter and a second longitudinal diameter, the first longitudinal diameter may be substantially smaller than the second longitudinal diameter, wherein the first longitudinal diameter may be at the second end and the second longitudinal diameter may be longitudinally displaced from the second end and more toward the collar.

The barb-type portion may be located within a region of the first longitudinal length diameter.

The fluid reservoir connector and/or the humidification chamber connector may comprise a lumen or passageway for enabling fluid communication between the first end and the second end.

A force required to separate the fluid reservoir connector and/or the humidification chamber connector and the conduit may be at least about 20 N.

A force required to separate the fluid reservoir connector and/or the humidification chamber connector and the conduit may be at least about 30 N.

A force required to separate the fluid reservoir connector and/or the humidification chamber connector and the conduit may be about 20 N to about 110 N.

A force required to separate the fluid reservoir connector and/or the humidification chamber connector and the conduit may be about 30 N to about 100 N.

A force required to separate the fluid reservoir connector and the fluid reservoir may be at least about 20 N.

A force required to separate the fluid reservoir connector and the fluid reservoir may be at least about 30 N.

A force required to separate the fluid reservoir connector and the fluid reservoir may be about 20 N to about 200 N.

A force required to separate the fluid reservoir connector and the fluid reservoir may be about 30 N to about 100 N.

A force required to separate the conduit and the humidification chamber and/or fluid reservoir may be at least about 20 N.

A force required to separate the conduit and the humidification chamber and/or fluid reservoir may be at least about 30 N.

A force required to separate the conduit and the humidification chamber and/or fluid reservoir may be about 20 N to about 110 N.

A force required to separate the conduit and the humidification chamber and/or fluid reservoir may be about 30 N to about 100 N.

A force required to separate or disconnect the humidification assembly, or one or more components of the humidification assembly, may be at least about 20 N.

A force required to separate or disconnect the humidification assembly, or one or more components of the humidification assembly, may be at least about 30 N.

A force required to separate or disconnect the humidification assembly, or one or more components of the humidification assembly, may be about 20 N to about 200 N.

A force required to separate or disconnect the humidification assembly, or one or more components of the humidification assembly, may be about 30 N to about 100 N.

A separation of one or more components of the humidification assembly may cause destruction of at least one component of the humidification assembly.

The conduit may be flexible.

The conduit may be at least partially transparent.

The conduit may be formed of polyvinyl chloride (PVC).

The conduit may have a length for creating sufficient pressure to supply fluid from the fluid reservoir to the humidification chamber.

The fluid pressure may be insufficient to damage components of the humidification assembly, when in use.

The conduit may be at least 0.1 m in length.

The conduit may be about 0.3 m to 3 m in length.

The conduit may be about 0.5 m to 2 m in length.

The conduit may be about 0.6 m in length.

The conduit may be a conduit assembly, and may comprise of one or more conduits connected together.

The connection between these one or more conduits may be permanent.

The fluid reservoir may be permanently connected with a first conduit.

The humidification chamber may be permanently connected with a second conduit.

The first and second conduits may be permanently connected together, for example by a permanent joint, or by a connector such as the connector described above, thereby providing a permanently connected fluid reservoir and humidification chamber. There may be other conduit portions provided between the first and second conduit portions, each of said conduits or conduit portions being permanently connected together.

The fluid reservoir may comprise a tapered base or floor which leads to an outlet port or to a fluid outlet corresponding to, or associated with, a fluid reservoir connection point.

The fluid reservoir may comprise at least one inlet port capable of being sealed or closed, said inlet port for receiving a supply of humidification fluid.

The inlet port may be capable of being sealed or closed by a lid or a cap.

The lid or cap may be attached or retained to the fluid reservoir or the inlet port by a flexible arm or strap.

The fluid reservoir may be charged or re-charged with said humidification fluid (e.g. fillable and/or refillable) via the inlet port.

The fluid reservoir may comprise an attachment for securing to or hanging the fluid reservoir from a support.

The attachment may be a closed loop or an open hook.

The attachment may be provided as a separate component part, and/or may be integrally formed with the inlet port and/or the lid or cap.

The fluid reservoir may comprise at least partially transparent or translucent walls to enable visual inspection of the humidification fluid level.

The fluid reservoir may be a flexible receptacle.

The fluid reservoir may have a capacity of about 0.25 L to about 5 L.

The fluid reservoir may have a capacity of about 0.5 L to about 2 L.

The fluid reservoir may have a capacity of about 1 L.

The fluid reservoir may comprise an index or indices for indicating a humidification fluid level or a fluid volume.

The fluid reservoir may be a water bag.

The fluid reservoir may be formed of polyvinyl chloride (PVC).

The humidification assembly may further comprise a controller to control a rate of flow of a fluid to be received from the fluid reservoir, or a rate of flow of a fluid to be discharged or released from the fluid reservoir, or controls a rate of flow of a fluid to be admitted to the humidification chamber.

The humidification chamber may comprise indices or an index or a visual guide for indicating fluid level within said humidification chamber.

The humidification assembly or kit may be sterile or may be provided as sterile component parts of a sealed container or receptacle.

The humidification assembly or kit as described above may be configured for use or coupling or provided as part of a high flow respiratory support system or a high flow respiratory support circuit comprising a flow source (or flow generator) and a patient interface.

The humidification assembly or kit as described above may be configured for use or coupling or provided as part of a respiratory support system or a respiratory support circuit comprising a flow source (or flow generator) and a patient interface.

The patient interface may be of a sealing or a non-sealing type.

The patient interface may be one of: a nasal cannula, an oro-nasal mask, a mouth-piece, a full-face mask, or a nasal cannula comprising of a sealing or non-sealing nasal prong or prongs.

The respiratory support system and/or the humidification assembly or kit as described above may comprise a dry line or a conduit for directing a source of respiratory gas to a humidification chamber or for provision to a respiratory breathing circuit or system.

The respiratory support system and/or the humidification assembly or kit as described above may comprise an inspiratory conduit.

The respiratory support system and/or the humidification assembly or kit as described above may comprise an expiratory conduit.

The humidification assembly or kit may be for multi-patient use.

The humidification assembly or kit may have a pre-determined period of in-use capability.

The humidification assembly or kit may be for use for about 24 hours.

The humidification assembly or kit may be provided with an expiry period of about 24 hours of use.

The fluid reservoir may be configured to retain a volume or quantity of humidification fluid.

The humidification chamber and the fluid reservoir may be provided as separate component parts.

The fluid reservoir may be configured to be charged, or re-charged, or is rechargeable, with a supply of humidification fluid.

The connection may be a hermetic connection.

At least one, or each, of said established connection(s) may be made by one or more of:
a barbed-type connector,
a structural formation providing for a physical inter-connect,
an interference or friction fit or push-fit,
an adhesive,
solvent bonding,
heat sealing,
a threaded connection.

The humidification fluid may be a liquid.

The humidification fluid may be liquid water.

The humidification fluid may be water, hypotonic water, and/or distilled water.

Water contained in or to be added to the fluid reservoir may be substantially pure, and/or may not contain solutes.

Fluid contained in or to be added to the fluid reservoir may be sterile.

Water contained in or to be added to the fluid reservoir may be distilled water.

In a sixth aspect there is provided a respiratory support system for delivering humidified gas to a user or patient, comprising:
a flow source,
a patient interface, and
the humidification assembly or kit as described in any of the above aspects or features as disclosed herein,
wherein the humidification chamber is in fluid communication with the flow source and the patient interface.

The patient interface may be for single-patient use.

The flow source may be operable to provide a gas flow of at least about 5 LPM.

The flow source may be operable to provide a gas flow of about 5 LPM to about 150 LPM.

The flow source may be operable to provide a gas flow of about 20 LPM to about 90 LPM.

The flow source may be operable to provide a gas flow of about 40 LPM to about 70 LPM.

The respiratory support system may be configured for use or coupling or provided as part of a high flow respiratory support system or a high flow respiratory support circuit comprising a flow source (or flow generator) and a patient interface.

The respiratory support system may be configured for use or coupling or provided as part of a respiratory support system or a respiratory support circuit comprising a flow source (or flow generator) and a patient interface.

The patient interface may be of a sealing or a non-sealing type.

The patient interface may be one of: a nasal cannula, an oro-nasal mask, a mouth-piece, a full-face mask, or a nasal cannula comprising of a sealing or non-sealing nasal prong or prongs.

The respiratory support system and/or the humidification assembly or kit as described above may comprise an expiratory conduit.

The assembly or kit may comprise a first assembly or first kit providing components for continued use with multiple patients, and a second assembly or second kit providing components for individual use with an individual patient.

The first assembly or first kit may comprise the assembly or kit as described above.

The first assembly or first kit may comprise:
a fluid reservoir,
a humidification chamber,
optionally a conduit for providing of a fluid connection between the fluid reservoir and the humidification chamber,
optionally a dry line or a conduit for directing a source of respiratory gas to a humidification chamber or for provision to a respiratory breathing circuit or system, and
optionally an inspiratory conduit.

The second assembly or second kit may comprise:
a patient interface,
a filter, or an in-line filter, for connection between the patient interface and an inspiratory conduit of a first assembly or first kit.

The second assembly or second kit may comprise an expiratory conduit.

Any of the aforementioned features or embodiments or aspects may be combined with one or more of the other features or embodiments or aspects as described herein.

The term “hypotonic” fluid used in this specification may include distilled water that may or may not be sterile. Hypotonic water has substantially no salts and has an osmolarity of substantially 0 mOsm/Kg. In contrast, saline or IV fluids (that are isotonic) may have an osomolarity of about 300 mOsm/Kg. An example of the water that could be used to fill a fluid reservoir as described herein would be known as “hypotonic USP sterile water”, however it will be understood that any suitable humidifier fluid could be used for delivery from a fluid reservoir to a humidification chamber for humidification purposes.

The terms ‘conduit’ and ‘tubing’ as used in this specification and claims are intended to broadly mean, unless the context suggests otherwise, any member that forms or provides a lumen for directing a flow of liquid or gases. For example, a conduit or conduit portion may be part of a humidification device, or may be a separate conduit attachable to a humidification device to provide a flow of fluid or a fluid communication.

The terms “comprising” and/or “including” as used in this specification and claims means “consisting at least in part of”. When interpreting each statement in this specification and claims that includes the term “comprising” and/or “including”, features other than that or those prefaced by the term may also be present. Related terms such as “comprise” and “comprises”, “include” and “includes” are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

As used herein the term “and/or” means “and” or “or”, or both.

As used herein “(s)” following a noun means the plural and/or singular forms of the noun.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

The disclosure consists in the foregoing and also envisages constructions of which the following gives examples only. Features disclosed herein may be combined into new embodiments of compatible components addressing the same or related inventive concepts. For example, connection of one illustrated embodiment may be combined with fluid container or humidification chamber of another illustrated embodiment.

### BRIEF DESCRIPTION OF THE FIGURES

Preferred embodiments of the disclosure will be described by way of example only and with reference to the following drawings.

FIG. 1 schematically shows a respiratory therapy system incorporating a humidification assembly, for example the humidification assembly or kit of FIG. 2, 3 or 7.

FIG. 2 shows a perspective view of an embodiment of a humidification assembly of the present disclosure.

FIG. 3 shows components of an embodiment of a humidification assembly of the present disclosure.

FIGS. 4A and 4B show perspective and front views of a connector of an embodiment of the present disclosure.

FIGS. 5A and 5B show perspective views of a fluid reservoir of an embodiment of the present disclosure.

FIG. 6 shows a perspective view of a fluid reservoir of an embodiment of the present disclosure.

FIG. 7 shows an embodiment of a humidification assembly of the present disclosure.

### DETAILED DESCRIPTION

Various embodiments are described with reference to the Figures. Throughout the Figures and specification, the same reference numerals may be used to designate the same or similar components, and redundant descriptions thereof may be omitted.

Hypotonic water already exists in bottles in theatre for other reasons. The invention of this disclosure provides for an apparatus, system or assembly in which this water can be used to fill/refill a water bag. The water bag is to be provided in permanent fluid communication with a humidifier chamber.

The present disclosure is intended to minimise, reduce or ameliorate, the risk of providing hypotonic water to a patient, and/or directing an isotonic solution to a humidification chamber. With the disclosed humidification assembly or kit, medical personnel may have to make a conscious and/or intentional effort to incorrectly administer the wrong fluid bag. This is a problem not recognized with current fluid (water and IV) bags and humidifier chambers/infusion pumps that are used in an operating theatre/room, where optionality in what to connect to is valued over permanence of connection. Moreover, incorrectly administering the wrong bag may have serious implications for a patient, such that there exists a real patient and equipment safety risk in a busy and/or stressful theatre environment.

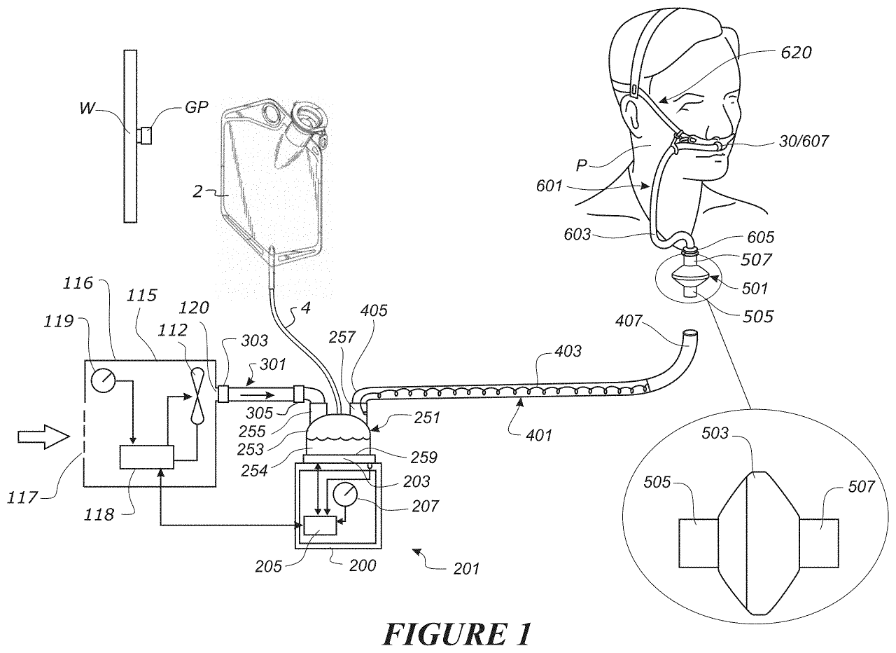

FIG. 1 illustrates a humidifying respiratory circuit. A patient P is receiving humidified and pressurised gases through a nasal cannula assembly of a patient interface 601 that is operatively connected to a humidified gases transportation pathway or inspiratory conduit 401 via a filter 501. The inspiratory conduit 401 in turn is connected to a humidifier 200 (including humidifier chamber 251) that is supplied with gases from a blower 115 or other appropriate gases supply means via a gases delivery conduit 301. The gases delivery conduit is a ‘dry’ conduit; that is, it is positioned upstream of the humidifier. Headgear 620 is provided to support and retain the patient interface against the patient's face.

The inspiratory conduit 401 is connected to the outlet 257 of the humidifier chamber 251 which contains a volume of liquid such as water. Humidifier chamber 251 may be formed from a plastics material and may have a highly heat conductive base 259 (for example an aluminium base) which is in direct contact with a heater plate 203 of humidifier 200.

The humidifier 200 is provided with control means or electronic controller 205 which may comprise a microprocessor based controller executing computer software commands stored in associated memory. Gases flowing through the inspiratory conduit 401 are passed to the patient by way of an optional filter 501 and patient interface 601.

The controller 205 receives input from sources such as user input means or dial 207 through which a user of the device may, for example, set a predetermined required value (pre-set value) of humidity or temperature of the gases supplied to patient P. In response to the user set humidity or temperature value input via dial 207 and other possible inputs such as internal sensors that sense gases flow or temperature, or by parameters calculated in the controller, controller 205 determines when (or to what level) to energise heater plate 203 to heat the water within humidifier chamber 251. As the volume of water within humidifier chamber 251 is heated, water vapour begins to fill the volume of the chamber above the water's surface and is passed out of the humidifier chamber 251 outlet port 257 with the flow of gases (for example air) provided from a gases supply means or blower 115 which enters the chamber through gases inlet port 255. It should be noted that it is possible to obtain the relationship between the humidity of the gases in humidifier chamber 251 and the temperature of the heater plate 203. Accordingly, it is possible to utilise the heater plate temperature in an algorithm or a look-up table to determine the humidity of the gases.

The blower 115 may be provided with a variable speed pump or fan 112 which draws air or other gases through the blower inlet 117. The speed of variable speed pump or fan 112 may be controlled by a further control means or electronic controller 118 (or alternatively the function of this controller 118 could be carried out by the other controller 205) in response to inputs from controller 205 and a user set predetermined required value (pre-set value) of pressure or fan speed or flow rate via dial 119 or other input device. Alternatively, the gases may be provided from a wall supply; i.e. a wall gas port GP in a wall W.

A housing 116 of the blower is provided with an outlet port 120. An inlet port 303 of the gases delivery conduit 301 and the outlet port 120 of the blower are provided with complementary coupling features to connect the outlet port 120 with the inlet port 303, and to provide a gases flow path therethrough. The complementary coupling features may in part be provided by an adapter insert.

In some configurations, rather than using a blower 115, gases flow may be obtained from some other source(s) of gas. For example, in some configurations, source(s) of gas may comprise one or more containers of compressed air and/or another gas and one or more valve arrangements adapted to control the rate at which gases leave the one or more containers. As another example, in some configurations, gases may be obtained from an oxygen concentrator. The system may also include a supplementary gases source to provide an air and supplementary gas mixture. For example, the supplementary gas might be O2. In some configurations, the apparatus may be adapted to deliver a high flow therapy.

“High flow therapy” as used in this disclosure may refer to delivery of gases to a patient at a flow rate of greater than or equal to about 5 or 10 litres per minute (5 or 10 LPM or L/min).

In some configurations, ‘high flow therapy’ may refer to the delivery of gases to a patient at a flow rate of about 5 or 10 LPM to about 150 LPM, or about 15 LPM to about 95 LPM, or about 20 LPM to about 90 LPM, or about 25 LPM to about 85 LPM, or about 30 LPM to about 80 LPM, or about 35 LPM to about 75 LPM, or about 40 LPM to about 70 LPM, or about 45 LPM to about 65 LPM, or about 50 LPM to about 60 LPM. For example, according to those various embodiments and configurations described herein, a flow rate of gases supplied or provided to an interface or via a system or from a flow source, may comprise, but is not limited to, flows of at least about 5, 10, 20, 30, 40, 50, 60, 70, 80, 90, 100, 110, 120, 130, 140, 150 L/min, or more, and useful ranges may be selected to be any of these values (for example, about 20 to about 90 LPM, about 40 to about 70 LPM, about 40 to about 80 LPM, about 50 to about 80 LPM, about 60 to about 80 LPM, about 70 to about 100 L/min, about 70 to 80 L/min).

Gases delivered may comprise a percentage of oxygen. In some configurations, the percentage of oxygen in the gases delivered may be about 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

High flow therapy has been found effective in meeting or exceeding the patient's inspiratory flow, to increase oxygenation of the patient and/or reduce the work of breathing. Additionally, high flow therapy may generate a flushing effect in the nasopharynx such that the anatomical dead space of the upper airways is flushed by the high incoming gas flows. This creates a reservoir of fresh gas available of each and every breath, while minimising re-breathing of carbon dioxide, nitrogen, etc.

The humidifier 200 has a humidifier base comprising a housing 201 with the heater 203, the controller 205 that is coupled to the heater, and the user input device 207 to enable a user to turn on and off the humidifier and to select a desired temperature to be provided by the heater. The user input device 207 may for example be a button, switch, or touch screen display. The heater 203 may comprise one or more heating elements.

The humidifier base is configured to receive the humidifier chamber 251. The humidifier chamber 251 comprises a housing 253 defining an internal liquid reservoir 254, an upstream gases inlet port 255 in fluid/pneumatic communication with the liquid reservoir, a downstream gases outlet port 257 in fluid/pneumatic communication with the liquid reservoir, and a base 259. The base 259 is arranged to be positioned on or above the heater 203 to heat liquid in the liquid reservoir. The base may comprise a flange 261 that projects outwardly from an adjacent portion of the housing 253, to assist with locating the humidifier chamber in position on the humidifier base.

The gases inlet port 255, the liquid reservoir 254, and the gases outlet port 257 are in fluid/pneumatic communication to provide a gases flow path from the gases inlet port 255, through or past the liquid reservoir, to the gases outlet port 257 to heat and humidify gases travelling along the gases flow path.

The humidifier chamber 251 may be any suitable chamber that holds suitable liquid for use in humidifying gases, such as water for example. The humidifier chamber 251 may be a manual fill chamber, and may be filled through a liquid inlet port 263. Alternatively and preferably, the humidifier chamber 251 may be an automatically filling chamber, and liquid may be fed to the humidifier chamber from a fluid reservoir/liquid container, bag, or other liquid source. The humidifier chamber may comprise a float valve in the liquid reservoir, the float valve configured to control flow of liquid form the liquid container into the liquid reservoir.

A gases delivery conduit 301 is located upstream of the humidifier chamber 251. The gases delivery conduit 301 is in fluid/pneumatic communication with the humidifier chamber 251 or is configured to be placed in fluid/pneumatic communication with the humidifier chamber upstream of the humidifier chamber; i.e. with the humidifier chamber 251 downstream of the gases conduit 301. The gases delivery conduit 301 is configured to receive one or more gases from a source of gas and deliver the gas(es) to the gases inlet port 255 of the humidifier chamber.

The gases delivery conduit 301 has an upstream gases inlet port 303 at one end of the conduit, and a downstream gases outlet port 305 at the opposite end of the conduit. The gases inlet port 303 and the gases outlet port 305 are in fluid/pneumatic communication to provide a gases flow path from the gases inlet port 303 through the gases delivery conduit to the gases outlet port 305. The gases outlet port 305 of the gases delivery conduit and the gases inlet port 255 of the humidifier chamber 251 may comprise complementary coupling features, to enable the gases delivery conduit 301 to be coupled to the humidifier to provide fluid/pneumatic communication between the gases delivery conduit 301 and the humidifier chamber 251. The complementary coupling features of the gases outlet port 305 of the gases delivery conduit 301 and the gases inlet port 255 of the humidifier chamber 251 may be disconnectable from each other to enable the gases delivery conduit 251 to be decoupled from the humidifier chamber 251. Alternatively, the complementary coupling features may be permanently or semi-permanently coupled.

The gases inlet port 303 of the gases delivery conduit may be provided with coupling feature(s) to enable the gases delivery conduit to be coupled to the source of gas.

An inspiratory conduit 401 extends from the humidifier chamber 251 to link the humidifier to a patient interface 601 via an optional in-line filter 501. The inspiratory conduit 401 may comprise a conduit heater 403 adapted to heat gases passing through the conduit 401. The heater 403 will help minimise or prevent the formation of condensation in the inspiratory conduit, which could otherwise occur due to a temperature differential between the interior of the conduit wall and exterior of the conduit wall. In other configurations the conduit heater 403 may not be present. The inspiratory conduit 401 comprises an upstream gases inlet port 405 at one end of the conduit and a downstream gases outlet port 407 at the opposite end of the conduit, with the conduit defining a gases flow path from the gases inlet port 405 to the gases outlet port 407.

The humidifier chamber 251 is in fluid/pneumatic communication with the gases inlet port 405 upstream of the inspiratory conduit 401 or is configured to be placed in fluid/pneumatic communication with the gases inlet port 405 upstream of the inspiratory conduit; i.e. with the inspiratory conduit positioned downstream of the humidifier chamber 251. The gases outlet port 257 of the humidifier chamber 251 and the gases inlet port 405 of the inspiratory conduit 401 may comprise complementary coupling features, to enable the inspiratory conduit 401 to be coupled to the humidifier to provide fluid/pneumatic communication between the humidifier chamber 251 and the inspiratory conduit 401. The complementary coupling features of the gases outlet port 257 of the humidifier chamber 251 and the gases inlet port 405 of the inspiratory conduit 401 may be disconnectable from each other to enable the inspiratory conduit 401 to be decoupled from the humidifier chamber 251. Alternatively, the complementary coupling features may be permanently or semi-permanently coupled.

The inspiratory conduit 401 will typically have a longer length than the gases delivery conduit 301.

The optional filter 501 comprises a generally cylindrical filter housing 503 with an enlarged central body portion. A leading edge of the enlarged central body portion comprises a tapering wall that terminates at an upstream gases inlet port 505, and a trailing edge of the enlarged central body portion terminates at a downstream gases outlet port 507. The gases inlet port 505 and gases outlet port 507 are in fluid/pneumatic communication via the central body portion. The filter may be a high-efficiency particulate arrestance (HEPA) filter. The enlarged central portion of the filter housing contains suitable filtration material. For example, the filtration material may comprise pleated paper, nano-fibers, or any other suitable filtration material, including sock filters, stacked disc filters, spiral filters, block(s) of filter material, a disc or discs of filter material with streams of filter material to free flow from or off the disc in fluid flow. The filter captures and prevents downstream passage therethrough of particulates, bacteria and/or other infectious material from the inspiratory conduit to the patient, and also captures and prevents upstream passage therethrough of bacteria and/or other infectious material from the patient to the inspiratory conduit.

The gases inlet port 505 of the filter 501, if present, and the gases outlet port 407 of the inspiratory conduit 401 may comprise complementary coupling features, to enable the inspiratory conduit to be coupled to the filter to provide fluid/pneumatic communication between the inspiratory conduit and the filter. The complementary coupling features of the gases inlet port 505 of the filter, if present, and the gases outlet port 407 of the inspiratory conduit 401 are disconnectable from each other to enable the inspiratory conduit 401 to be decoupled from the filter 501.

In one configuration, the complementary coupling features between the gases outlet port 407 of the inspiratory conduit 401 and the gases inlet port 505 of the filter comprise a 22 mm medical connection or 22 mm medical taper connection. An electrical connector may be provided on the gases inlet port of the inspiratory conduit. The electrical connector may provide for a power connection to a heater wire of the inspiratory conduit, or therethrough to any associated components with inspiratory conduit or other parts of the kit or assembly. The electrical connector may also all provide connection to signal, communication or sensor wires.

The optional filter 501 is in fluid/pneumatic communication with the patient interface 601 upstream of the patient interface or is configured to be placed in fluid/pneumatic communication with the patient interface 601 upstream of the patient interface 601; i.e. with the patient interface located downstream of the filter. In one configuration, the filter 501 is coupled to the patient interface 601 or is configured to be coupled to the patient interface 601.

The patient interface 601 comprises a patient interface gases conduit 603 with an upstream gases inlet port 605 at one end of the conduit. The opposite downstream end of the patient interface gases conduit 603 is in fluid/pneumatic communication with a patient cannula 30/607 to deliver gases from the patient interface gases conduit 603 to a patient P.

In one configuration, the gases outlet port 507 of the optional filter 501 and the gases inlet port 605 of the patient interface gases conduit comprise complementary coupling features to enable the filter 501 to be coupled to the patient interface 601 to provide fluid/pneumatic communication between the filter and the patient interface gases conduit, with the filter in-line with a gases flow path through the patient interface gases conduit. The complementary coupling features may be disconnectable from each other to enable the filter to be decoupled from the patient interface gases tube of the patient interface. Alternatively, the complementary coupling features may be permanently or semi-permanently coupled.

In one configuration, the complementary coupling features between the gases outlet port 507 of the filter 501 and the gases inlet port 605 of the patient interface 601 comprise a 22 mm medical taper connection.

In an alternative configuration, the patient interface 601 comprises a patient interface gases conduit 603, and the filter 501 is integrally formed with the patient interface gases conduit to provide fluid/pneumatic communication between the filter 501 and the patient interface gases conduit 603, with the filter in-line with a gases flow path through the patient interface gases conduit. That is, the filter and patient interface may be an integrated unit.

In another example embodiment without a filter, the gases outlet port 407 of the inspiratory conduit 401 connects to the gases inlet port 605 of the patient interface gases conduit 603. The gases outlet port 407 of the inspiratory conduit 401 and the gases inlet port 605 of the patient interface gases conduit 603 may comprise complementary coupling features, to enable the inspiratory conduit to be coupled to the patient interface conduit to provide fluid/pneumatic communication between the inspiratory conduit and the patient interface conduit. The complementary coupling features of the gases outlet port 407 of the inspiratory conduit and the gases inlet port 605 of the patient interface gases conduit 603 are disconnectable from each other to enable the inspiratory conduit 401 to be decoupled from the patient interface.

In one configuration, the complementary coupling features between the gases outlet port 407 of the inspiratory conduit 401 and the gases inlet port 605 of the patient interface gases conduit 603 comprise a 22 mm medical connection or 22 mm medical taper connection.

The patient interface 601 is shown to be a nasal cannula, although it should be understood that in some configurations, other patient interfaces may be suitable. For example, in some configurations, the patient interface may comprise a sealing or non-sealing interface, and may comprise a nasal mask, an oral mask, an oro-nasal mask, a full face mask, a nasal pillows mask, a nasal cannula, an endotracheal tube, a tracheostomy tube, a combination of the above or some other gas conveying system. In an embodiment, the patient interface 601 comprises a non-sealing interface such as a nasal cannula, which allows gases to be exchanged with the environment. For example, the non-sealing cannula allows carbon dioxide to be removed and/or cleared from the patient's airways while the patient receives flow therapy from the system. Further, in an embodiment, the patient interface is in the form of a nasal interface, such that the system does not interfere with other oral airway equipment and/or devices, for example, a tracheal tube in an intubation procedure. Accordingly, the patient may continue to receive flow therapy throughout the intubation procedure.

The patient interface gases conduit 603 forms a first gas lumen defined by a tubular wall. The first gas lumen is adapted to receive gases from the respiratory therapy system, via the inspiratory conduit 401 and filter 501 shown in FIG. 1, and channel the gases to the patient P.

The illustrated first gas lumen is defined at least in part by a wall within which gases can be channelled.

The first gas lumen may optionally comprise a reinforcement element adapted to strengthen and/or add rigidity to the first gas lumen to prevent deformation or collapse of the first gas lumen arising due to the application of forces against the first gas lumen. The reinforcement element may include a number of structures, including but not limited to plastic or metallic reinforcing beads that lie in or on the wall of the first gas lumen. Alternatively, the lumen may, in some configurations, comprise weakened sections, or sections which are unable to self-maintain their flow path or a fluid flow passage, to allow for a mask to seal over the patient interface, such as a nasal cannula or nasal mask, and to reduce or prevent the flow of gases to the patient interface.

The first gas lumen 603 is in fluid/pneumatic communication with a flow manifold. The flow manifold receives gases from the first gas lumen 603 and passes them to one or more nasal delivery elements (e.g. the nasal prongs of a nasal cannula, not shown). The one or more nasal delivery elements extend outwardly from the flow manifold. The one or more nasal delivery elements are adapted to be non-sealingly positioned in one or more nares of the patient P.

As shown, in one example, the patient interface 601 can comprise two nasal delivery elements adapted to be positioned one in each of the patient's nares. Each nasal delivery element may be shaped or angled such that it extends inwardly towards a septum of the patient's nose.

Additionally, each nasal delivery element may be shaped or angled such that a tip of each nasal delivery element points, in use, towards a back of the head of the patient P. In the embodiment shown in FIG. 1, the flow manifold receives flow from one lateral side of the flow manifold (e.g. with respect to an imaginary vertical plane bisecting the face of the patient P) and channels flow to each of the nasal delivery elements. In other configurations, the patient interface 601 may comprise greater (for example, three or four) or fewer (for example, one) nasal delivery element.

In other configurations, each nasal delivery elements can have different properties. For example, one of a pair of nasal delivery elements can be relatively long and the other nasal delivery element can be relatively short. In some configurations, the flow manifold may be configured to receive flow from two lateral sides of the flow manifold (e.g. from a ‘left’ and ‘right’ of the flow manifold when instead of just the ‘left’ of the flow manifold as seen in FIG. 1). In some such configurations, multiple gas lumens may be used to provide for pneumatic communication between the flow manifold and the respiratory therapy system. In some configurations, the flow manifold may be configured to receive flow from a non-lateral side of the flow manifold (e.g. from a ‘bottom’ or ‘top’ of the flow manifold).

In other configurations, as noted, the manifold may be a separately attachable component to be attached to the body of the interface, such as a nasal cannula or nasal mask. Such a manifold may be an entirely separate component able to be removed from attachment to the interface, or it may be de-attached or disconnected from an operational position to allow for a re-orientation of the manifold (and associated supply conduit) relative to the interface. For example, the manifold may be of a push-fit type arrangement to be push-fitted into a connection with the interface body, or may be of a swivel-type connection with the body of the interface allowing for a re-orientation of the manifold. A re-orientation allows for the supply conduit to be positioned to a left or a right side of the interface (and therefore changed from one side to another of the patient). This may allow for an improved convenience or arrangement of components in a system delivering gas to the patient. For example, if those assisting in a medical procedure need access to the patient from one particular side, then the manifold can be re-oriented and the supply tube re-positioned so as to extend from a different side of the patient. Such an arrangement allows for a relatively unobtrusive application of a patient interface and its associated components away from medical specialists.

The patient interface when in the form of a nasal cannula may utilise a headgear in the form of a strap that can be bifurcated (i.e. a line of weakness or other split arrangement can be configured) to allow for the headgear or a strap thereof to be reconfigured from a single strap arrangement into a bifurcated strap arrangement.

The patient interface when in the form of a nasal cannula may utilise a pair of side arms extending from the main body (to which the manifold is to be put into connection with). The side arms may comprise of features allowing for the retention or securement or positioning of a gas supply tube to the side arm (to prevent the gas supply tube from uncontrollably moving about).

The patient interface 601 may further comprise mounts and/or supports, e.g., cheek supports, for attaching and/or supporting the gas lumen 603 and/or cannula 30/607 on the patient's face. For example, a releasable connection system may be utilised to position or locate the interface upon the patent's face, yet allow for a relatively rapid removal or re-positioning of the interface if necessary.

The patient interface gases conduit or gas lumen may be a short section of tube or conduit. For example, the patient interface gases conduit or gas lumen may be about 20 cm to about 50 cm long, or about 25 cm to about 40 cm long, or about 30 cm to about 35 cm long, or may be about 32 cm long.

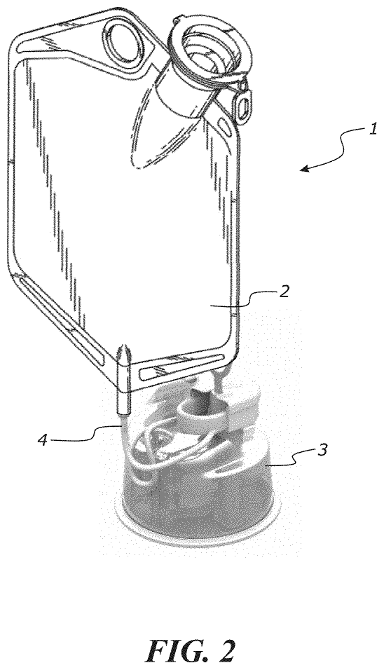

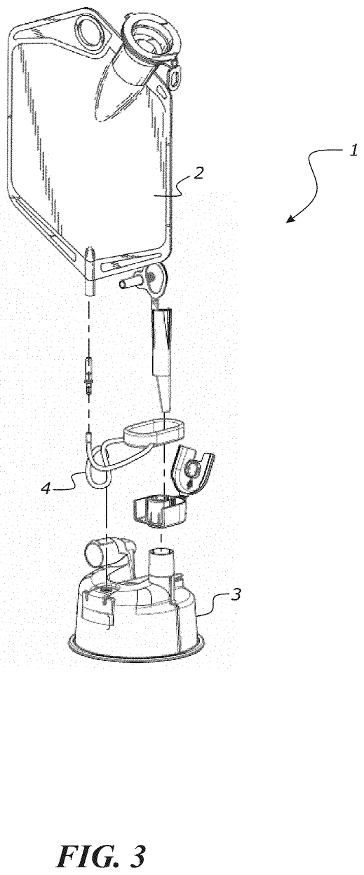

FIGS. 2 and 3 illustrate example embodiments of a humidification assembly or kit 1 for use in a respiratory support system such as that shown in FIG. 1. The humidification assembly or kit 1 may deliver humidified gases to a user or patient via the respiratory support system as illustrated in FIG. 1, and can include a fluid reservoir 2 for providing a source of humidification fluid, a humidification chamber 3 (such as humidifier chamber 251 in FIG. 1) for receiving a source of humidification fluid, and a connection to transfer the humidification fluid from the fluid reservoir 2 to the humidification chamber 3.

The fluid reservoir 2 and the humidification chamber 3 are in fluid connection or communication with each other, with a connection established between each of the fluid reservoir 2 and the humidification chamber 3 to provide humidification fluid via the fluid communication. The fluid reservoir 2 and the humidification chamber 3 may be in fluid connection or communication even when the humidification assembly or kit 1 is not providing humidified gases to a user or patient. The fluid connection, fluid communication or fluid pathway may be provided by a device such as a conduit 4, tubing, or other form of fluid connection or communication. One end of the device may be connected to the fluid reservoir 2, and another end may be connected to the humidification chamber 3. Alternatively, in other embodiments there may be a direct connection established between a fluid reservoir and a humidification chamber.

The connection may be a permanent connection. The connection may have a pre-determined force of retention or resistance to disconnection. The connection may be provided with a force of retention or resistance to disconnection such that when the connection is disconnected, it is destructively severed, for example upon application of a disconnection force above a pre-determined force of retention.

Additionally, a part or component of the humidification assembly or kit 1 may be destroyed or damaged when the established permanent connection is subject to a disconnection force above a pre-determined force of retention.

The connection between each of the fluid reservoir 2 and the humidification chamber 3, may be a one-time, or non-reusable connection, or may be an enduring connection. Alternatively, the connection or fluid communication may be established as a one-time, secured, resilient, or a resistant (to disconnection) or an enduring connection to provide a fluid pathway or fluid connection between the fluid reservoir 2 and the humidification chamber 3 that cannot be reused or easily disconnected.

Turning back to FIGS. 2 and 3, the pre-determined force of retention and/or resistance to disconnection of these example humidification assemblies may be a force of retention provided between the conduit 4 and the humidification chamber 3, and/or the conduit 4 and the fluid reservoir 2.

Alternatively, the connection provided by the conduit 4 between the fluid reservoir 2 and humidification chamber 3, may be effectively permanent, such that removal in the normal scheme of use would be difficult, and/or would result in deformation or damage of one of the components of the assembly.

As outlined above, the established connection may be configured such that, a conduit 4 providing for the fluid communication is resistant to, or unable to be disconnected, or where disconnection results in damage to the fluid communication. Separation or disconnection of the established connection may serve to destructively sever the connection, or separation or disconnection of the established connection may be destructive of one or more components or parts which formed the established connection. Separation or disconnection of the connection may serve to prevent a subsequent re-establishment of a connection.

The connection between the conduit 4 and humidification chamber 3, and/or the conduit 4 and the fluid reservoir 2, may be made by one or more of:
a barbed-type connector,
a structural formation providing for a physical inter-connect,
an interference or friction fit or push-fit,
an adhesive,
solvent bonding,
heat sealing,
threaded connection,
any combination of the above.

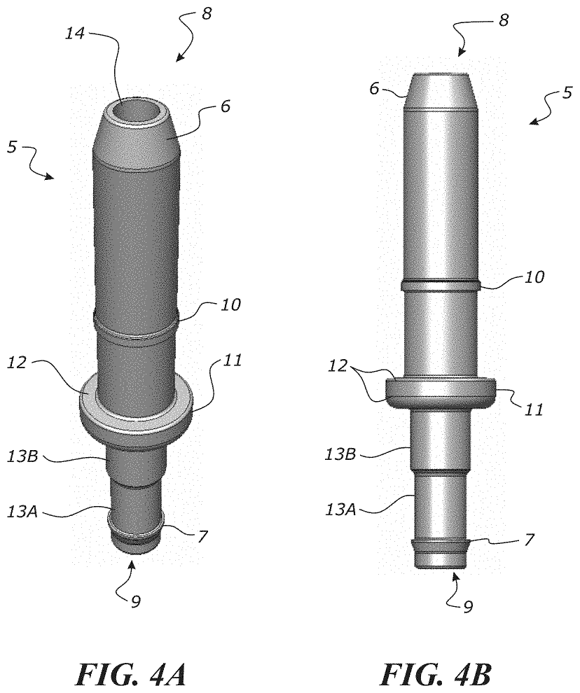

FIGS. 4A and 4B show an example embodiment of such a connector 5, which may be used to connect the conduit 4 and fluid reservoir 2 or humidification chamber 3 of FIGS. 2 and 3. The connector 5 in FIGS. 4A and 4B utilises a barb type-connection, but it should be apparent that any other suitable connector may be used to provide a one-time connection capability.

The connector 5 in FIGS. 4A and 4B may be a fluid reservoir connector 5, disposed at a fluid reservoir end of a conduit 4, and configured to connect to a fluid outlet of a fluid reservoir.

Additionally, the connector 5 in FIGS. 4A and 4B may be a humidification chamber connector 5, disposed at a humidification chamber end of a conduit 4, and configured to connect to a fluid inlet of a humidification chamber 3. Alternatively, the conduit 4 may be adhered, e.g. glued into connection with the humidification chamber 3.

Therefore, in an example embodiment a conduit 4 may comprise a fluid reservoir connector 5 at a fluid reservoir end of the conduit 4, and/or a humidification chamber connector 5 at a humidification chamber end of the conduit 4. One or both of these connections may be the abovementioned connection, configured to be permanent, and/or resistant to disconnection.

Any of the abovementioned connections may be hermetic connections. In an example, this provides a hermetic connection between the humidification chamber 3 and the fluid reservoir 2, via the conduit 4.

To achieve the permanent, one-time, or enduring connection described above, the connector 5 of FIGS. 4A and 4B, representing any of the abovementioned connections, may comprise a lead-in portion 6 substantially at or toward one end of said connector 5. The lead-in portion 6 may facilitate the connection, and may be a tapered region, shaped so as to facilitate the establishment of connection.

The tapered region may be tapered to provide for a relatively narrowed neck of a relatively smaller diameter, which may allow receipt of a conduit 4, or allow the connector 5 to be received by the fluid reservoir 2 or the humidification chamber 3 3. The tapered region may facilitate ease of connection, by guiding the connector 5 into the fluid reservoir 2 or humidification chamber 3, or the conduit 4 onto the connector 5.

The connector 5 may be substantially flexible or substantially conformable or non-rigid, which could alleviate stress on the connection formed by this connector 5. Alternatively, the connector 5 may be substantially inflexible or substantially non-conformable or substantially rigid, which may facilitate ease of establishment of the connection.

Alternatively, the connector 5 may comprise both substantially conformable, and substantially non-conformable parts, which may include soft or flexible parts along with hard or rigid parts, or a hinge region.

The connector 5 may be formed of polypropylene, or silicon, or any other suitable harder or softer material to facilitate the desired permanent connectability.

In an example embodiment, the connector 5 may be a fluid reservoir connector 5, and may be a substantially longitudinal component comprising a first end 8 configured to be received by the fluid reservoir, and a second end 9 configured to be received by the conduit 4. A lead-in portion 6 may be disposed at or toward the first end 8, and a radially outward portion 7, for example a barb-type arrangement or portion, may be disposed at or toward the second end 9.

The radially outward portion 7, may be configured to retain the conduit 4 once a connection is established. The portion 7 may be radially or annularly continuous, or may be radially or annularly discontinuous.

The connection may additionally comprise at least one radially outward projection 10, which may extend from an outer surface of the fluid reservoir connector 5. The radially outward projection 10 may be located intermediate of the lead-in portion 6 and the second end 9, and may comprises of a barb-type arrangement, configured to retain a fluid reservoir once the connection is established. A leading edge or leading surface of the radially outward projection 10 may be leading with respect to an edge or surface of the projection(s) which is/are to preferentially make contact with said fluid reservoir. The at least one radially outward projection 10 may be radially continuous, or radially discontinuous. There may be several radially outward projections 10 located intermediate the lead-in portion 6 and the second end 9, to provide additional security of retention of a connection.

The connector 5 may further comprise a collar 11, which may be located intermediate of the radially outward projection 10 and the second end 9. The collar 11 can provide for at least two seating surfaces 12, a first of said seating surfaces 12 provided to substantially face toward the first end 8 and a second of said seating surfaces 12 provided to substantially face toward the second end 9. Each seating surface may be provided as a continuous or discontinuous surface.

The collar 11 may be an annular collar 11, where the collar 11 extends radially outwardly from the outer surface of the fluid reservoir connector 5. The collar 11 may be dimensioned to have a longitudinal thickness configured to separate a terminal end of the conduit 4 from a fluid reservoir connection point. The collar 11 may be a separate component part of the connector 5, or may be a thickened band region of the connector 5.

The at least one radially outward projection 10 may extend outwardly of the outer surface to a substantially lesser extent or diameter than the collar 11. Collar 11 may therefore provide a seating surface for a corresponding part of a fluid reservoir. Similarly, the radially outward portion 7 of the connector 5 may extend outwardly to a lesser extent than the collar 11. Collar 11 may therefore also provide a seating surface for the conduit end. Alternatively, radially outward projection 10 and portion 7 may provide engagement features, optionally to corresponding engagement features of a fluid reservoir and/or a conduit. Collar 11 may have greater diameter than radially outward projection 10 and portion 7, and may therefore provide seating surfaces to corresponding features of a fluid reservoir and/or a conduit.

The radially outward portion 7 may be located substantially at or towards the second end 9, and may be located intermediate of the collar 11 and the second end 9, providing for secure retention of a conduit 4.

The external diameter of the connector 5 between the second end 9 and the collar 11 may comprise two different longitudinal length diameters 13A, 13B. The first longitudinal diameter 13A may be substantially smaller than the second longitudinal diameter 13B, and the first diameter 13A may be at the second end 9, whereas the second diameter 13B may be longitudinally displaced from the second end 9 and more toward the collar 11. The first longitudinal diameter 13A may assist in providing and/or improving the permanent coupling and connection between a conduit and the connector 5, e.g. via increasing adhesion between contact surfaces, components or parts. The second longitudinal diameter 13B may, alongside the radially outward portion 7, or as a replacement to the radially outward portion 7, provide for a friction fit to provide secure retention of a conduit 4. In this configuration, the radially outward portion 7 may be located within a region of the first longitudinal length diameter 13A. In alternative embodiments, radially outward portion 7 may be located within a region of the second longitudinal diameter 13B. In some embodiments, there may be several radially outward portions 7 located within both regions 13A and 13B, providing additional security of retention of a connection.

The connector 5 of FIGS. 4A and 4B may comprise a lumen 14 or passageway for enabling fluid communication between the first end 8 and the second end 9, thus providing for a fluid connector 5 for use at a fluid reservoir and/or humidification chamber end of a conduit 4.

The force required to overcome the pre-determined force of retention outlined above, in order to separate the fluid reservoir connector 5 and/or the humidification chamber connector 5 and the conduit 4 may be at least about 20 N, or about 30 N, or may be between about 20 N to about 110 N, or about 30 N to about 100 N.

Similarly, the force required to separate the fluid reservoir connector 5 and the fluid reservoir 2 may be at least about 20 N, or about 30 N, or may be between about 20 N to about 200 N, or about 20 N to about 110 N, or about 30 N to about 100 N. In some embodiments, the force required to separate the fluid reservoir connector 5 and fluid reservoir 2 is up to about 200 N.

Similarly, the force required to separate the conduit 4 and the humidification chamber 3 may be at least about 20 N, or about 30 N, or may be between about 20 N to about 110 N, or about 30 N to about 100 N.

Generally, the force required to separate or disconnect the humidification assembly or kit, or to separate or disconnect one or more components of the humidification assembly, would be more, preferably substantially more, than the forces experienced during normal use (for example forces experienced during normal use could be 20 N or less). The force required to separate or disconnect the humidification assembly or kit, or to separate or disconnect one or more components of the humidification assembly, may be at least about 20 N, or at least about 30 N, or may be between about 20 N to about 110 N, or about 30 N to about 100 N. Separation of any or all connections of the humidification assembly or kit may be achieved only upon the application of a force, along one or more axes of engagement of the connection, having a magnitude which exceeds the forces experienced during normal use of the assembly or kit by a magnitude of at least 100%, at least 200%, at least 300%, at least 400%, at least 500%, at least 600%, at least 700%, at least 800%, at least 900%, at least 1000%, or at least 1100%. In some embodiments, the magnitude of force experienced to separate or disconnect one or more components of the assembly or kit may be up to 2000% of the forces experienced during normal use of the assembly or kit. It will be understood that a pre-determined force of retention as outlined above, could be any suitable force for resisting disconnection of the humidification assembly or kit. A separating or disconnection force equal to or more than this pre-determined force of retention would be required to separate the components of the humidification assembly or kit. In certain embodiments, a separating or disconnection force more than this pre-determined force of retention would be required to separate the components of the humidification assembly or kit.

Any separation of one or more components of the humidification assembly or kit can cause destruction of at least one component of the assembly or kit. For example, a separation or disconnection of the established connection may serve to destructively sever the connection. This may include damage or destruction to the conduit 4, the connector 5 between the fluid reservoir 2 and the conduit 4, and/or the connector 5 between the humidification chamber 3 and the conduit 4, and/or connection points on the fluid reservoir 2 or humidification chamber 3. Additionally, a separation or disconnection of an established connection of the humidification assembly may serve to prevent or preclude a subsequent re-establishment of a connection. Put another way, any of the abovementioned connections may be of a one-time connection capability.

The conduit 4 forming the fluid connection or communication between the fluid reservoir 2 and the humidification chamber 3 may be flexible, and may be at least partially transparent. The conduit 4 may be formed of polyvinyl chloride (PVC), or any other suitable material to provide a flexible or semi-flexible fluid connection. Alternatively, the fluid connection may be rigid, and may be opaque.

In an example embodiment, the conduit 4 has a length that is sufficient for creating sufficient pressure to supply fluid from the fluid reservoir 2 to the humidification chamber 3. However, the fluid pressure may be insufficient to damage components of the humidification assembly, when in use.

The conduit 4 may be at least 0.1 m in length, however it could be between about 0.3 m to 3 m in length, or about 0.5 m to 2 m in length. In an example embodiment, the conduit 4 length is about 0.6 m, which may provide the abovementioned sufficient pressure to supply fluid from the fluid reservoir 2 to the humidification chamber 3, without causing component damage due to excess fluid pressure. However, it will be understood that the effects of conduit 4 or tubing length, diameter, and material will vary the required conduit length and supply pressure accordingly. The supply pressure may also be affected by the fluid reservoir, including the fluid reservoir outlet, and the humidification chamber and its components.

The device or conduit 4 connecting the fluid reservoir 2 and the humidification chamber 3 may be formed as a conduit assembly, and may comprise one or more conduits connected together. The connection between these one or more conduits may be permanent. For example, the fluid reservoir 2 may be permanently connected with a first conduit 4, and the humidification chamber 3 may be permanently connected with a second conduit 4. The first and second conduits may be permanently connected together, for example by a permanent joint, or by a connector such as connector 5, thereby providing a permanently connected fluid reservoir 2 and humidification chamber 3. It will be understood that any suitable number or configuration of fluid connection or communication devices, such as conduit 4, could be provided in permanent connection with each other to achieve a connection between a fluid reservoir and humidification chamber that is secured as described herein. In some embodiments, there may be other conduit portions provided between the first and second conduit portions, each of said conduits or conduit portions being permanently connected together.

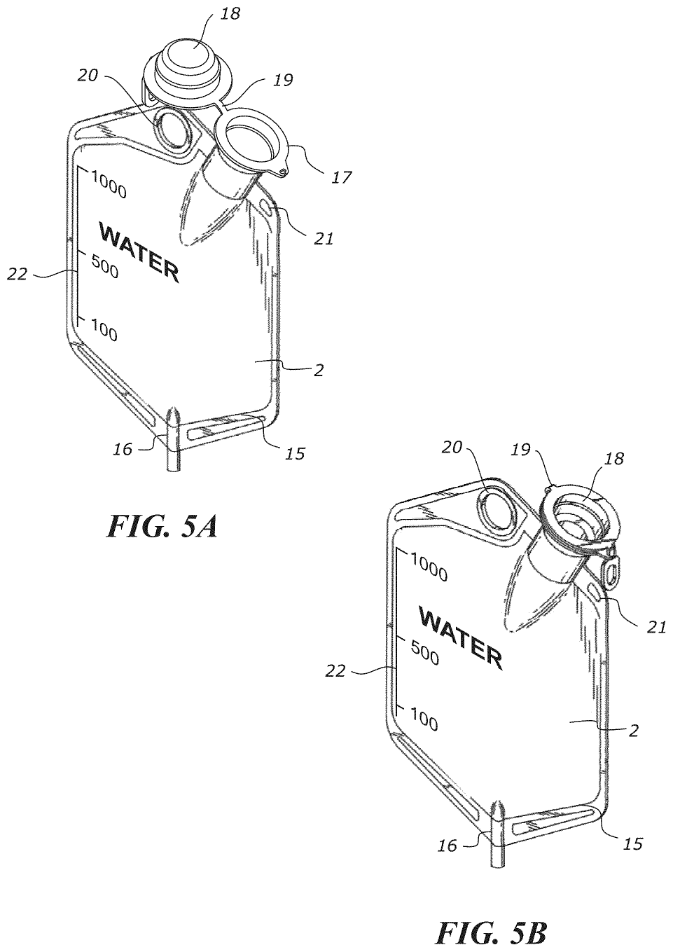

FIGS. 5A and 5B show an example embodiment of a fluid reservoir 2 as described above. The fluid reservoir 2 may comprise a tapered base 15 or floor, which may lead to an outlet port or to a fluid outlet 16 corresponding to, or associated with, a fluid reservoir connection point. The outlet port or fluid outlet 16 may be configured to receive the connector 5 of FIGS. 4A and 4B as described above, and/or may be configured to directly receive a conduit 4.

The fluid reservoir 2 may comprise at least one inlet port 17, which may capable of being sealed or closed. The inlet port 17 may be configured to receive a supply of humidification fluid, and the fluid reservoir 2 may be charged or re-charged with said humidification fluid (e.g. fillable and/or refillable) via the inlet port 17.

The inlet port 17 may be capable of being sealed or closed by a lid or a cap 18. The lid or cap 18 may be attached or retained to the fluid reservoir 2, and/or the inlet port 17, by a flexible arm or strap 19.

The fluid reservoir 2 may comprise an attachment 20 for securing to or hanging the fluid reservoir 2 from a support. The attachment 20 could be a closed loop, as illustrated in FIGS. 5A and 5B, or an open hook (not shown), or any other suitable attachment point, such as through holes 21 in FIGS. 5A and 5B.

The attachment 20 may be provided as a separate component part, or may be integrally formed with the inlet port 17 and/or the lid or cap 18 (not shown). The attachment 20 may also be formed as an integral part of the fluid reservoir 2, as shown in FIGS. 5A and 5B. Alternatively, the attachment 20 may be an integrally formed part of the fluid reservoir 2 structure, or may form a part of the inlet 17 and/or lid/cap 18 structure, or may be provided as a separate hanger portion, which can be attached to the fluid reservoir 2 to provide another facility for hanging the fluid reservoir from a support.

The fluid reservoir 2 may comprise at least partially transparent or translucent walls, or a transparent section, to enable visual inspection of the humidification fluid level. The fluid reservoir 2 may comprise an index or indices 22 for indicating a humidification fluid level or a fluid volume contained within the fluid reservoir 2. Additionally, the humidification chamber 3 as described herein may also, or alternatively, comprise indices or an index or a visual guide for indicating fluid level within the humidification chamber 3. The indices could also be formed on the conduit 4.

The fluid reservoir 2 may be a flexible receptacle (for example, the flexible receptacle may be a bag, such as a water bag). Alternatively, the fluid reservoir 2 may comprise a rigid or semi-rigid container. In either case, the fluid reservoir 2 may be formed of a plastic, or other suitable material for containing fluid, for example polyvinyl chloride (PVC).

The fluid reservoir 2 is configured to retain a volume or quantity of humidification fluid, and may have a capacity of about 0.25 L to about 5 L, or about 0.5 L to about 2 L. In an example embodiment, the capacity of the fluid reservoir 2 is about 1 L.

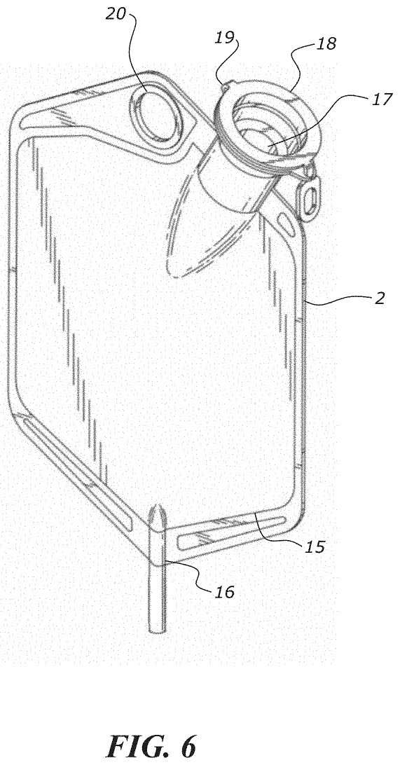

FIG. 6 illustrates an example of a fluid reservoir 2 suitable for use with the humidification assembly or kit as described herein. This fluid reservoir 2 includes a single attachment point 20 integrated into the fluid reservoir 2, and an off-centre inlet port 17 for filling and refilling the fluid reservoir 2. Alternatively, the inlet port 17 could be provided in the centre of the fluid reservoir 2, for example taking the place of attachment point 20. An attachment point could be provided as an integral part of such a central inlet port, or one or more attachment point(s) could be provided at either side of such a central inlet port, optionally these attachment point(s) could be through holes integrally formed as part of the fluid reservoir.

The humidification assembly or kit 1 as described above may include a controller to control a rate of flow of humidification fluid to be received from the fluid reservoir 2, to the humidification chamber 3. The controller may be used to control a rate of flow of fluid to be discharged and/or released from the fluid reservoir 2, or to control a rate of flow of fluid to be delivered and/or admitted to the humidification chamber 3. The controller may be an integral part of the conduit 4, fluid reservoir 2, and/or humidification chamber 3, or may be a separate component.

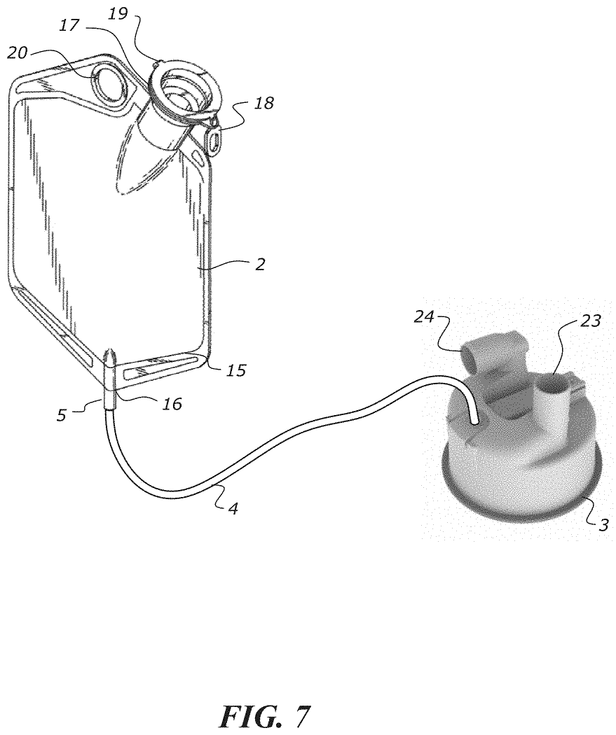

FIG. 7 shows an example embodiment of a humidification assembly or kit 1 as described herein, comprising fluid reservoir 2, humidification chamber 3, and conduit 4. The fluid reservoir 2 includes tapered base 15 and fluid outlet 16, along with inlet port 17 comprising a lid 18, strap 19, and attachment 20. In this example embodiment, connector 5 is shown providing a one-time and/or permanent connection between conduit 4 and fluid reservoir 2. The other end of conduit 4 is permanently attached to humidification chamber 3 by an adhesive or friction fit. Humidification chamber 3 also includes gases inlet 23, for connection to a dry line or other source of un-humidified breathing gases, and gases outlet 24, for connection to an inspiratory conduit for transmission of humidified breathing gases to a patient. Gases inlet 23 and gases outlet 24 may be equivalent to gases inlet port 255 and outlet 257 as shown in FIG. 1.

The humidification assembly or kit may be sterile, or at least provided as sterile component parts of a sealed container or receptacle. For example, the humidification assembly or kit may be provided for use in a surgery or theatre situation, and provided with an anaesthesia kit in a sealed receptacle to be opened and used for patient humidification during a procedure.

The humidification assembly or kit may be configured for use or coupling or provided as part of a respiratory support system, or a respiratory support circuit comprising a flow source (or flow generator) and a patient interface (such as a cannula). The respiratory support system could be a high flow system.

The patient interface could be of a sealing or a non-sealing type, and could be one of a nasal cannula, an oro-nasal mask, a mouth-piece, a full-face mask, or a nasal cannula comprising of a sealing or non-sealing nasal prong or prongs.

The respiratory support system and/or the humidification assembly or kit may also include a dry line or a gases conduit for directing a source of respiratory gas to a humidification chamber 3, or for provision to a respiratory breathing circuit or system.

The respiratory support system and/or the humidification assembly or kit may also include an inspiratory conduit, and/or an expiratory conduit.

The humidification assembly or kit may be designed for multi-patient use, and may have a pre-determined period of in-use capability, for example, the humidification assembly or kit may have a use lifetime of about 24 hours, and may be provided with an expiry period of about 24 hours of use.

The respiratory support system may comprise a first assembly or first kit providing components for continued use with multiple patients, and a second assembly or second kit providing components for individual use with an individual patient.

The first kit may comprise the humidification assembly or kit 1 as described herein, including a fluid reservoir 2, a humidification chamber 3, a conduit 4 for providing of a fluid pathway or fluid connection between the fluid reservoir 2 and the humidification chamber 3, optionally a dry line or a gases conduit for directing a source of respiratory gas to a humidification chamber 3 or for provision to a respiratory breathing circuit or system, and optionally an inspiratory gases conduit for receiving humidified gases from the humidifier outlet and directing them to a patient interface component such as a patient interface, or an intermediate connector such as a y-piece. The first kit may be provided as a clean or sterile kit.

The second kit may comprise a patient interface, and a filter, or an in-line filter, for connection between a patient interface. This may facilitate the multi-patient usability of the humidification assembly or kit, by providing a single patient use patient interface, with a filter to ensure a connected humidification assembly or kit remains clean between patients. Further, the fluid reservoir 2 provides a reservoir of humidification fluid to replenish the humidification fluid in the humidification chamber 3 that has been lost or used up during operation of the respiratory support system—which provides for use with multiple patients—while at the same time reducing or eliminating the need to constantly re-fill or re-charge the humidification chamber 3 manually. The second kit may be provided as a clean or sterile kit.

It will be understood that any of the abovementioned kits or assemblies, or any of their component parts, may be provided together in any combination as a kit or assembly. For example, as a kit for use in theatre, where some of the components may be for multiple patient use, and some may be for individual patient use, and where some or all of the components of the kit may be provided clean or sterile, as described above.

The humidification chamber 3 and the fluid reservoir 2 of the humidification assembly or kit 1 may be provided as separate component parts, and when connected by a user (for example via a conduit 4), the connection may be sufficient to resist disconnection, and/or may be permanent and/or a one-time connection.

The fluid reservoir 2 may be configured to receive, (and to deliver to a humidification chamber 3 via a conduit 4 and one-time connection), liquid water, for example distilled water, or fluid that is sterile, substantially pure, and/or does not contain solutes. As an example, sterile water may already be present in a theatre or surgery environment for other uses, and this water may be used to fill the fluid reservoir 2 via the inlet port 17.

The respiratory support system described above may be configured to deliver humidified gases to a user or patient, and may comprise a flow source, a patient interface, and the humidification assembly or kit as described herein. Additionally, the humidification chamber 3 may be in fluid communication with the flow source and the patient interface.

The flow source may be operable to provide a gas flow rate of at least about 5 LPM, or about 20 LPM to about 90 LPM, or about 40 LPM to about 70 LPM, or up to about 150 LPM.

Similarly, the flow source may be operable to provide and/or the respiratory support system is operable to deliver to a patient, a gas flow rate of greater than or equal to about 5 or 10 litres per minute (5 or 10 LPM). In other configurations, the gas flow rate may be about 5 or 10 LPM to about 150 LPM, or about 15 LPM to about 95 LPM, or about 20 LPM to about 90 LPM, or about 25 LPM to about 85 LPM, or about 30 LPM to about 80 LPM, or about 35 LPM to about 75 LPM, or about 40 LPM to about 70 LPM, or about 45 LPM to about 65 LPM, or about 50 LPM to about 60 LPM. For example, according to those various embodiments and configurations described herein, a flow rate of gases supplied or provided to an interface or via a system or from a flow source, may comprise, but is not limited to, flows of at least about 5, 10, 20, 30, 40, 50, 60, 70, 80, 90, 100, 110, 120, 130, 140, 150 L/min, or more, and useful ranges may be selected to be any of these values (for example, about 20 to about 90 L/min, about 40 to about 70 L/min, about 40 to about 80 L/min, about 50 to about 80 L/min, about 60 to about 80 L/min, about 70 to about 100 L/min, about 70 to 80 L/min).

This invention discloses permanent connections between a fluid reservoir 2 (e.g. a water bag) and a humidification chamber 3 via a conduit 4 (e.g. a tubing); i.e. a permanent or one-time connection provided between a fluid reservoir 2 and one end of a conduit 4, and a permanent or one-time connection between a humidification chamber 3 and another end of the conduit 4.

The arrangement described above is an atypical set-up and serves the purpose of ensuring only water is used in a humidification system, as the user is unable to accidentally spike (connect to) a bag containing a fluid other than water. This problem is not recognized in the art, which generally prefers flexibility in allowing connections to many different types of fluids. Accordingly, applicants have surprisingly found that permanently connecting these components provides advantages such as improved patient safety and ease of use for the medical staff in the operating theatre.

Testing was done to ensure the force required to separate:
The fluid reservoir connector and the fluid reservoir.
The fluid reservoir connector and/or the humidification chamber connector and the conduit.
   is at least 30 N.

This force requirement was found to be satisfied when the connections were unconditioned, transport conditioned, duration of use conditioned and shelf life conditioned.

The method for testing involved mounting the humidification chamber in a test rig with the permanent connections being axially aligned with a load cell that was attached to the fluid reservoir. Peak tension was then recorded during a 500 mm/minute extension until failure. The failure mode was also recorded.

The failure modes included a break along the interconnecting conduit, a break at the humidification chamber connection or the barb adaptor slipping from the interconnecting tube.

When the samples were unconditioned, the peak tension or force ranged from about 50 N to about 70 N. There was about 50% failure due to the barb adapter slipping from the conduit and 50% failure from a break in the connection at the humidification chamber. There was no failure in conduit breaking.

When the samples were conditioned to simulate transportation stresses, the peak force ranged from about 50 N to about 70 N. There was about 95% failure due to the barb adapter slipping from the conduit and 5% failure from a break in the connection at the humidification chamber. There was no failure in conduit breaking.

When the samples were conditioned to simulate the stresses during use (in this test, the duration of use was 7 days) the peak force ranged from about 50 N to about 80 N. There was about 55% failure due to the barb adapter slipping from conduit and 45% failure from a break in the connection at the humidification chamber.

When the samples were conditioned at 60° C. to the equivalent of 2 years storage/shelf-life, the peak force ranged from about 40 N to about 70 N. There was 100% failure due to the barb adapter slipping from conduit. There was no failure by either a break in the conduit or a break in the connection at the humidification chamber. The shelf-life conditioned samples were estimated to have a 5.6-8.3 N (drop of 9.0-13.3%) effect on the mean retention force but the samples were still above the minimum required 30 N.

Where, in the foregoing description reference has been made to integers or components having known equivalents thereof, those integers are herein incorporated as if individually set forth.

In the foregoing description, the terms ‘permanent’ and ‘permanently’ are intended to include the terms ‘one-time’, ‘secured’, ‘resilient’, ‘resistant (to disconnection)’, ‘non-releasable’, ‘durable’ and/or ‘enduring’, used in regard to the connection established between any or all of a fluid reservoir, a conduit, and a humidification chamber. The terms ‘permanent’ or ‘permanently’ are intended to describe a connection that, when formed, is not separable in the normal use of equipment containing these components. Put another way, by use of permanent connection, or the related terms outlined herein, it is meant that said connection cannot be undone by the application of forces which are experienced during normal use of the assembly or kit, and there is no mechanism for releasing the connection, or disengaging any protrusions or formations that may enable the permanence of the connection, without either damaging one or more component parts of the assembly, or applying a force between components that exceeds a pre-determined threshold force along an axis of engagement of a connection.

Although the present disclosure has been described in terms of certain embodiments, other embodiments apparent to those of ordinary skill in the art also are within the scope of this disclosure. Thus, various changes and modifications may be made without departing from the spirit and scope of the disclosure. For instance, various components may be repositioned as desired. Moreover, not all of the features, aspects and advantages are necessarily required to practice the present disclosure. Accordingly, the scope of the present disclosure is intended to be defined only by the claims that follow.

1-65. (canceled)

66. A humidification assembly for use in a respiratory support system for delivering humidified gas to a user or patient, comprising:
a humidification chamber configured for receiving a source of humidification fluid,
a fluid reservoir configured for providing the source of humidification fluid for supply to the humidification chamber,
a conduit provided as an interconnect between the humidification chamber and the fluid reservoir configured for providing the source of humidification fluid, and
wherein, a connection is provided with a pre-determined force of retention and/or resistance to disconnection between each of:
the conduit and the humidification chamber, and
the conduit and the fluid reservoir.

67. The humidification assembly of claim 66, wherein the connection is one or more of:
wherein the connection is configured such that, the or a conduit providing for the fluid communication is resistant to, or unable to be disconnected,
wherein the connection is configured such that disconnection results in damage to the fluid communication,
wherein separation or disconnection of the connection serves to destructively sever the connection,
wherein separation or disconnection of the connection is destructive of one or more components or parts which formed the connection,
wherein separation or disconnection of the connection serves to prevent a subsequent re-establishment of a connection.

68. The humidification assembly of claim 66, wherein a first end of the conduit is connected or coupled to the fluid reservoir, or a component connected to or coupled to the fluid reservoir, wherein a second end of the conduit is connected or coupled to the humidification chamber or a component connected to or coupled to the humidification chamber

69. The humidification assembly of claim 68, wherein the conduit is provided as the fluid connection between the fluid reservoir and the humidification chamber, and is configured for providing the source of humidification fluid.

70. The humidification assembly of claim 66, wherein the conduit comprises:
a fluid reservoir connector at a fluid reservoir end of said conduit, and
a humidification chamber connector at a humidification chamber end of said conduit, wherein each of said fluid reservoir connector and said humidification chamber connector provide separately made connections between the conduit and the fluid reservoir and the conduit and the humidification chamber.

71. The humidification assembly of claim 70, wherein a force required to separate either the fluid reservoir connector or the humidification chamber connector from the conduit is at least about 20 N.

72. The humidification assembly of claim 70, wherein a force required to separate the fluid reservoir connector and the fluid reservoir is at least 20 N.

73. The humidification assembly of any one of claim 66, wherein a force required to separate the conduit and the humidification chamber or to separate the conduit and the fluid reservoir is at least 20 N.

74. The humidification assembly of claim 66, wherein a force required to separate the conduit and the humidification chamber from the fluid reservoir is at least 20 N.

75. The humidification assembly of claim 66, wherein a force required to separate the conduit and the humidification chamber from the fluid reservoir is either between 20 N to 110 N or between 30 N to 100 N.

76. The humidification assembly of claim 66, wherein a force required to separate the humidification assembly, or one or more components of the humidification assembly, is at least 20 N.

77. The humidification assembly of claim 66, wherein a force required to separate the humidification assembly, or one or more components of the humidification assembly is either between 20 N to 200 N or between 30 N to 100 N.

78. The humidification assembly of claim 76, wherein a separation of one or more components of the humidification assembly causes destruction of at least one component of the humidification assembly.

79. The humidification assembly of claim 77, wherein a separation of one or more components of the humidification assembly causes destruction of at least one component of the humidification assembly.

80. The humidification assembly of claim 66, wherein the conduit has a length for creating sufficient pressure to supply fluid from the fluid reservoir to the humidification chamber.

81. The humidification assembly of claim 66, wherein the conduit is about 0.5 m to 2 m in length.

82. The humidification assembly of claim 66, wherein the fluid reservoir comprises at least one inlet port configured to be sealed or closed, wherein the at least one inlet port is configured to receive a supply of humidification fluid.

83. The humidification assembly of claim 66, wherein the fluid reservoir has a capacity of about 1 L.

84. The humidification assembly of claim 66, wherein the fluid reservoir is a water bag.

85. The humidification assembly of claim 66, wherein the humidification assembly further comprises a controller to control at least one of:
a rate of flow of a fluid to be received from the fluid reservoir,
a rate of flow of a fluid to be discharged or released from the fluid reservoir, and
a rate of flow of a fluid to be admitted to the humidification chamber.

86. The humidification assembly of claim 66, configured for use as part of a high flow respiratory support system or a high flow respiratory support circuit comprising a flow source and a patient interface.

87. The humidification assembly of claim 66, wherein the patient interface is of a non-sealing type.

88. A humidification assembly for use in a respiratory support system for delivering humidified gas to a user or patient, comprising:
a humidification chamber configured for receiving a source of humidification fluid;
a fluid reservoir configured for providing the source of humidification fluid for supply to the humidification chamber;
a conduit provided as an interconnect between the humidification chamber and the fluid reservoir configured for providing the source of humidification fluid;
wherein, a connection is provided as a permanent connection between each of the conduit and the humidification chamber and the conduit and the fluid reservoir;
wherein a force required to separate the connection between the fluid reservoir from the conduit is at least about 20 N;
wherein a force required to separate the connection between the humidification chamber from the conduit is at least 20 N; and
wherein a separation of one or more components of the humidification assembly causes destruction of at least one component of the humidification assembly.

89. A kit for use in a respiratory support system for delivering humidified gas to a user or patient, comprising:
a humidification chamber configured for receiving a source of humidification fluid, a fluid reservoir configured for providing the source of humidification fluid for supply to the humidification chamber,
a conduit provided as an interconnect between the humidification chamber and the fluid reservoir configured for providing the source of humidification fluid, and
wherein, a connection is provided with a pre-determined force of retention and/or resistance to disconnection between each of:
the conduit and the humidification chamber, and
the conduit and the fluid reservoir.
